(19) United States
(12) Patent Application Publication
(10) Pub. No.: US 2026/0248801 A1
(43) Pub. Date: Aug. 27, 2026

(54) HIGH-POTENCY BIASED BETA-2 ADRENERGIC RECEPTOR AGONIST FOR TREATING RESPIRATORY DISEASES

(71) Applicant: UNIVERSITY OF SOUTH FLORIDA, Tampa, FL (US)

(72) Inventors: Stephen Bryant Liggett, Tampa, FL (US); Donghwa Kim, Tampa, FL (US); Yu Chen, Tampa, FL (US); Jesper J. Madsen, Tampa, FL (US); Steven Sung-Chur An, New Brunswick, NJ (US)

(21) Appl. No.: 19/552,632

(22) Filed: Feb. 27, 2026

Related U.S. Application Data
(60) Provisional application No. 63/764,567, filed on Feb. 27, 2025.

(51) Int. Cl. A61K 31/538; A61P 11/08

(52) U.S. Cl. CPC A61K 31/538; A61P 11/08

(57) ABSTRACT

Respiratory diseases such as asthma and chronic obstructive pulmonary disease (COPD) are commonly treated with β₂-adrenergic receptor agonists that activate intracellular signaling pathways to induce airway relaxation. However, current therapies are limited by progressive receptor desensitization, or tachyphylaxis, largely attributed to β-arrestin recruitment. This desensitization reduces therapeutic efficacy over time and complicates long-term disease management, resulting in suboptimal outcomes for patients who rely on bronchodilators for daily symptom control. Disclosed herein are pharmaceutical compositions comprising biased agonists and their methods of use, which minimally activate recruitment of beta-arrestin2.

### CROSS-REFERENCE TO RELATED APPLICATIONS

This application claims the benefit of priority to U.S. Provisional Application No. 63/764,567, filed Feb. 27, 2025, the disclosure of which is incorporated herein by reference in its entirety.

### BACKGROUND

In obstructive lung diseases such as asthma and chronic obstructive pulmonary disease (COPD), active human airway smooth muscle (HASM) cellular contraction limits airflow, representing a major cause of morbidity and mortality. β₂-adrenergic receptors (β₂ARs) expressed on HASM cells are the targets for binding of therapeutically administered β-agonists, which relax the cells via a cyclic adenosine monophosphate-mediated mechanism. However, the HASM bronchodilator response to acute β-agonist is attenuated by receptor desensitization, with typical treatments of humans, or isolated HASM cells, leading to a loss of receptor function over time, clinically termed tachyphylaxis. As such, current treatments of obstructive lung diseases are suboptimal with current β-agonists because they evoke tachyphylaxis via the beta-arrestin system. There remains a need for improved agents for treating such obstructive lung disorders without inducing tachyphylaxis. Furthermore, there remains a need for improved β₂AR agonists that are selective for Gas that do not interact with β-arrestin. There also remains a need for improved methods of treating obstructive lung disorders, including asthma. The compositions and methods disclosed herein address these and other needs.

### SUMMARY

Disclosed herein are pharmaceutical compositions and methods for using such pharmaceutical compositions. In some examples, disclosed herein are pharmaceutical compositions comprising a biased β₂-adrenergic receptor agonist according to Formula I

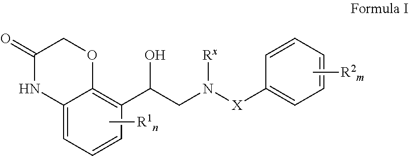

wherein all variables are defined herein. In some examples, disclosed herein are methods of treating obstructive lung disease without eliciting tachyphylaxis in a subject in need thereof, comprising administering a therapeutically effective amount of

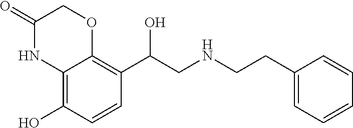 [SMILES: O=C1COc2c(C(O)CNCCc3ccccc3)ccc(O)c2N1]

or a pharmaceutically acceptable salt thereof.

The details of one or more embodiments of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 provides a structure of 5-hydroxy-8-(1-hydroxy-2-(phenethylamino)ethyl)-2H-benzo[b][1,4]oxazin-3 (4H)-one, which is termed compound A as described in the examples.

FIG. 2 provides graphs demonstrating the responses to ISO and Compound A in transfected CHO cells, measuring the stimulation of CAMP and beta-arrestin2. While ISO is a balanced agonist, compound A is biased towards CAMP and away from beta-arrestin2. Results are mean±/−SE of 4 experiments. *, P<0.01 vs ISO at all doses.

FIG. 3 provides a graph demonstrating the physiological response in HASM cells to continuous exposure to ISO or Compound A. The balanced agonist ISO showed a loss of relaxation over time. However, the response to 2 concentrations of the biased agonist Compound A remained stable. Data are mean±/−SE from 4 experiments. *, P<0.01 vs ISO.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular examples only and is not intended to be limiting.

### Definitions

As used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

It should be noted that amounts and other numerical data can be expressed herein in a range format. It can be further understood that the endpoints of each of the ranges are significant both in relation to the other endpoint and independent of the other endpoint. It is also understood that there are a number of values disclosed herein and that each value is also herein disclosed as “about” that particular value in addition to the value itself. For example, if the value “10” is disclosed, then “about 10” is also disclosed. Ranges can be expressed herein as from “about” one particular value and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’ ‘about y’, and ‘about z’ as well as the ranges of ‘less than x,’ less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y′, and ‘greater than z.’ In addition, the phrase “about ‘x’ to ‘y’,” where ‘x’ and ‘y’ are numerical values, includes “about ‘x’ to about ‘y’.”

It is to be understood that such a range format is used for convenience and brevity and, thus, should be interpreted in a flexible manner to include not only the numerical values explicitly recited as the limits of the range but also all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Examples defined by each of these transition terms are within the scope of this disclosure.

A “control” is an alternative subject or sample used in an experiment for comparison purposes. A control can be “positive” or “negative.”

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Optional” or “optionally” means that the subsequently described event or circumstance may or may not occur, and that the description includes instances where said event or circumstance occurs and instances where it does not.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “detect” or “detecting” refers to an output signal released for the purpose of sensing of physical phenomenon. An event or change in environment is sensed and signal output released in the form of light.

As used herein, “enhance”, “enhanced”, “enhancement”, “enhancing”, and any grammatical variations thereof as used herein, refers to an act of intensifying, increasing, or further improving the quality, value, or extent of a biological function, composition, compound, cell, or tissue.

As used herein, “EC₅₀,” is intended to refer to the concentration or dose of a substance (e.g., a compound or a drug) that is required for 50% enhancement or activation of a biological process, or component of a process, including a protein, subunit, organelle, ribonucleoprotein, etc. EC₅₀ also refers to the concentration or dose of a substance that is required for 50% enhancement or activation in vivo, as further defined elsewhere herein. Alternatively, EC₅₀ can refer to the concentration or dose of compound that provokes a response halfway between the baseline and maximum response. The response can be measured in an in vitro or in vivo system as is convenient and appropriate for the biological response of interest. For example, the response can be measured in vitro using cultured muscle cells or in an ex vivo organ culture system with isolated muscle fibers. Alternatively, the response can be measured in vivo using an appropriate research model such as rodent, including mice and rats. The mouse or rat can be an inbred strain with phenotypic characteristics of interest such as chronic obstructive pulmonary disease (COPD) or asthma. As appropriate, the response can be measured in a transgenic or knockout mouse or rat wherein the gene or genes has been introduced or knocked-out, as appropriate, to replicate a disease process.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., agonizing activity towards β-arrestin). It is understood that this is typically in relation to some standard or expected value, in other words it is relative, but that it is not always necessary for the standard or relative value to be referred to. For example, “reduces agonizing activity towards β-arrestin” means reducing β-arrestin activation relative to a standard or a control.

As used herein, the terms “dose”, “dosage”, “unit dose”, “unit dosage”, “effective dose” and related terms refer to physically discrete units that contain a predetermined quantity of active ingredient calculated to produce a desired therapeutic effect (e.g., bronchodilation or relaxation of the airways). These terms are synonymous with the therapeutically effective amounts and amounts sufficient to achieve the stated goals of the methods disclosed herein.

As used herein, the terms “treat”, “treating”, and “treatment” have their ordinary and customary meanings, and include one or more of, ameliorating a symptom of an obstructive lung disease or condition in a subject, blocking or ameliorating a recurrence of a symptom of an obstructive lung disease or condition in a subject, decreasing in severity and/or frequency a symptom of an obstructive lung disease or condition in a subject. As used herein, “treatment” includes at least partially, and at least temporarily, relieving bronchoconstriction (or bronchospasm) or increasing bronchodilation, so that the patient or subject can breathe more easily. Treatment means ameliorating, blocking, reducing, decreasing or inhibiting by about 1% to about 100% versus a subject to which a pharmaceutical composition has not been administered. Preferably, the ameliorating, blocking, reducing, decreasing or inhibiting is about 100%, 99%, 98%, 97%, 96%, 95%, 90%, 80%, 70%, 60%, 50%, 40%, 30%, 20%, 10%, 5% or 1% versus a subject to which a pharmaceutical composition has not been administered.

As used herein, the terms “prevent”, “preventing”, and “prevention” have their ordinary and customary meanings, and include one or more of preventing a symptom of an obstructive lung disease or condition in a subject, blocking a recurrence of a symptom of an obstructive lung disease or condition in a subject, and decreasing in frequency a symptom of an obstructive lung disease or condition in a subject. As used herein, “prevention” includes at least partially, and at least temporarily, blocking bronchoconstriction (or bronchospasm) so that breathing is not inhibited in the patient or subject. The prevention may be protection of about 100%, 99%, 98%, 97%, 96%, 95%, 90%, 80%, 70%, 60%, 50%, 40%, 30%, 20%, 10%, 5% or 1% in the subject, versus a subject to which a pharmaceutical composition has not been administered. The prevention lasts at least about 1, 2, 3, 4, 5, 6, 7, 8, 9, 10, 11, 12, 13, 14, 15, 16, 17, 18, 19, 20, 21, 22, 23, or 24, or more, hours after administration of a pharmaceutical composition.

An “adjuvant” refers to a drug, molecule, substance, or a combination thereof that is used to increase the efficacy or potency of certain therapeutic agents, such as for example vaccines and/or antibodies. “Adjuvant(s)” are often at least one ingredient used in some vaccines that help create a stronger immune response in the host receiving said vaccine.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “pharmaceutically acceptable” component can refer to a component that is not biologically or otherwise undesirable, e.g., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Therapeutic agent” refers to any composition that has a beneficial biological effect. Therapeutic agent includes, but is not limited to, genes and gene products, peptides and proteins, nanoparticles, nanoemulsions, lipids, carbohydrates, and small molecule drugs. Beneficial biological effects include both therapeutic effects, e.g., treatment of a disorder or other undesirable physiological condition, and prophylactic effects, e.g., prevention of a disorder or other undesirable physiological condition (e.g., a respiratory disease). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising a therapeutic agent) refers to an amount that is effective to achieve a desired therapeutic result. In some examples, a desired therapeutic result is sustained airway relaxation without evidence of tachyphylaxis. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Expression” as used herein refers to the process by which information from a gene is used in the synthesis of a functional gene product that enables it to produce a peptide/protein end product, and ultimately affect a phenotype, as the final effect.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein can also include tautomeric forms. Tautomeric forms result from the swapping of a single bond with an adjacent double bond together with the concomitant migration of a proton. Tautomeric forms include prototropic tautomers, which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds provided herein can also include all isotopes of atoms occurring in the intermediates or final compounds. Isotopes include those atoms having the same atomic number but different mass numbers. For example, isotopes of hydrogen include hydrogen, tritium, and deuterium.

Also provided herein are salts of the compounds described herein. It is understood that the disclosed salts can refer to derivatives of the disclosed compounds wherein the parent compound is modified by converting an existing acid or base moiety to its salt form. Examples of the salts include, but are not limited to, mineral or organic acid salts of basic residues such as amines; alkali or organic salts of acidic residues such as carboxylic acids; and the like. The salts of the compounds provided herein include the conventional non-toxic salts of the parent compound formed, for example, from non-toxic inorganic or organic acids. The salts of the compounds provided herein can be synthesized from the parent compound that contains a basic or acidic moiety by conventional chemical methods. Generally, such salts can be prepared by reacting the free acid or base forms of these compounds with a stoichiometric amount of the appropriate base or acid in water or an organic solvent or in a mixture of the two. In various aspects, nonaqueous media like ether, ethyl acetate, alcohols (e.g., methanol, ethanol, isopropanol, or butanol), or acetonitrile (MeCN) can be used.

“R¹”, “R²”, and “X” are used herein as generic symbols to represent various specific substituents. These symbols can be any substituent, not limited to those disclosed herein, and when they are defined to be certain substituents in one instance, they can, in another instance, be defined as some other substituents.

The term “alkyl” refers to a radical of a straight-chain or branched hydrocarbon group having a specified range of carbon atoms (e.g., a “C₁₋₁₆ alkyl” can have from 1 to 16 carbon atoms). An alkyl group can be a saturated alkyl group or an unsaturated alkyl group, e.g., an alkyl group having one or more carbon-carbon double/triple bonds, e.g., an alkenyl or alkynyl group. Unless specified to the contrary, an “alkyl” group includes both saturated alkyl groups and unsaturated alkyl groups. The alkyl group can be substituted with one or more groups including, but not limited to, hydroxyl, halogen, acyl, alkyl, alkoxy, alkenyl, alkynyl, aryl, heteroaryl, aldehyde, amino, cyano, carboxylic acid, ester, ether, ketone, nitro, phosphonyl, silyl, sulfo-oxo, sulfonyl, sulfone, sulfoxide, or thiol, provided that the substituents are sterically compatible and the rules of chemical bonding and strain energy are satisfied. Examples of alkyl groups include, but are not limited to, methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, ethenyl, propenyl, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, and the like.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “haloalkyl” specifically refers to an alkyl group that is substituted with one or more halides (halogens; e.g., fluorine, chlorine, bromine, or iodine). When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. By way of example, a heteroC₁₋₆ alkyl (which may also be designated a C₁₋₆ heteroalkyl) group includes, but is not limited to, the following structures:

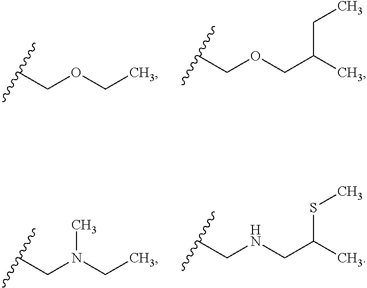

The term “heteroalkyl” preceded by a separate heteroatom refers to a heteroalkyl group bonded through the specified heteroatom. By way of example, an OC₁₋₆heteroalkyl group includes, but is not limited to, the following structures:

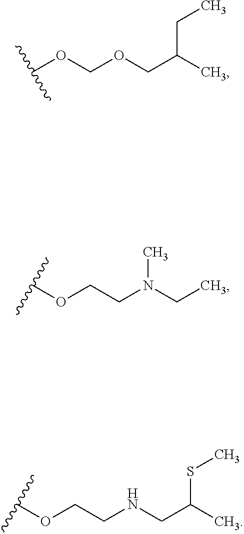

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

Affixing the suffix “-ene” to a group indicates the group is a polyvalent moiety, e.g., bonded to two or more groups. Alkylene is the polyvalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl (each of which parent groups as defined herein).

The term “alkoxy” refers to an alkyl group, as defined herein, covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In some examples, the cycloalkyl group is optionally substituted as described herein. The term “cycloalkyl” as used herein is not intended to be limited to monovalent radicals and may include polyvalent radical groups as appropriate, such as divalent, trivalent, tetravalent, pentavalent, and hexavalent cycloalkyl, and the like, based on the position and location of such groups in the compounds described herein as would be readily understood by the skilled person.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continues to designate the number of carbon atoms in the aryl ring system. Unless otherwise specified, each instance of an aryl group is independently unsubstituted (an “unsubstituted aryl”) or substituted (a “substituted aryl”) with one or more substituents.

The term “aryloxy” refers to an aryl group, as defined herein, covalently bound through an oxygen bridge (—O—).

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continues to designate the number of ring members in the heteroaryl ring system. “Heteroaryl” also includes ring systems wherein the heteroaryl ring, as defined above, is fused with one or more aryl groups, wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like), the point of attachment can be on either ring, e.g., either the ring bearing a heteroatom (e.g., 2-indolyl) or the ring that does not contain a heteroatom (e.g., 5-indolyl).

Exemplary heteroaryl and heterocyclyl rings include: benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyL cirrnolinyl, decahydroquinolinyl, 2H,6H~ 1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl.

Unless specified to the contrary, the alkyl, alkenyl, alkynyl, heteroalkyl, heteroalkenyl, heteroalkynyl, carbocyclyl, heterocyclyl, aryl, and heteroaryl groups defined herein (and the “ene” versions of said groups) may be substituted or unsubstituted. A substituted group includes a non-hydrogen substituent at a position where, in the unsubstituted version, a hydrogen atom would be found. Substituents include, but are not limited to, halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NR^{a}R^{b}, —NR^{a}C(═O)R^{b}, —NR^{a}C(═O)NR^{a}NR^{b}, —NR^{a}C(═O)OR^{b}, —NR^{a}SO₂R^{b}, —C(═O)R^{a}, —C(═O)OR^{a}, —C(═O)NR^{a}R^{b}, —OC(═O)NR^{a}R^{b}, —OR^{a}, —SR^{a}, —SOR^{a}, —S(═O)₂R^{a}, —OS(═O)₂R^{a} and —S(═O)₂OR^{a}. R^{a} and R^{b} in this context can be the same or different and independently hydrogen, halogen, hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, or heteroarylalkyl.

A dash (“-”) that is not between two letters or symbols is used to indicate a point of attachment for a substituent. For example, —(C—O)NH₂ is attached through the carbon of the keto (C═O) group.

As used herein, the designation of a polyvalent moiety without specifying the specific order of attachment is intended to cover all possible arrangements. By way of example, a compound represented by the formula:

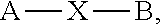

wherein X is NHC(═O) embraces both:

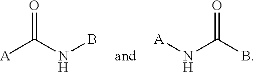

As used herein, a chemical bond depicted as [symbol] represents either a single, double, or triple bond, valency permitting. By way of example,

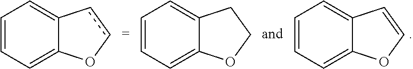

Unless stated to the contrary, a substituent drawn without explicitly specifying the point of attachment indicates that the substituent may be attached at any possible atom. For example, in a benzofuran depicted:

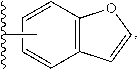

the substituent may be present at least at one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃. A group having the subscript ‘0’ is understood to represent a null group as well. By way of example, in the compound CH₃—(X)_{z}—CH₃, if X is CH₂ and z is 0, then the compound has the formula CH₃—CH₃.

A bracketed functional group with a subscripted variable should be understood to denote the number of repeated bracketed groups present. For example, a number that is selected from 0 or 1 should be interpreted as follows:

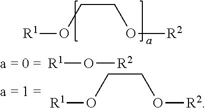

In some examples, two or more variable groups may together form a ring. It is understood that any depicted atoms separated from the identified groups will themselves form part of the ring:

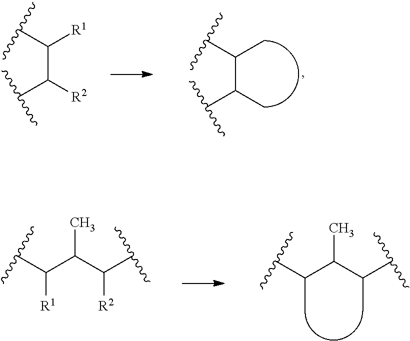

When the variable groups are substituted on an aromatic system, the new ring will be a fused ring, and unless specified to the contrary, may be either aromatic or non-aromatic, carbocyclic or heterocyclic:

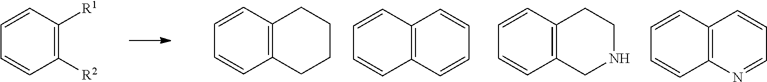

The ring may further be defined by the number of carbon atoms in the specific ring formed by the variable groups, which includes the atoms separating the variable groups:

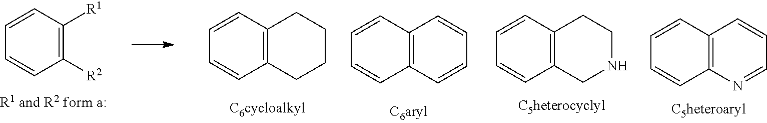

Each of the above results occurs when R¹ and R² together form a six-membered (or six-atom) ring. Other rings, including 3, 4, 5, 7, and 8-member rings, may also be formed and may be further limited by a specified number of carbon atoms. Although the singular “a ring” may be used to define the group, unless specified to the contrary, both monocyclic and polycyclic rings are possible:

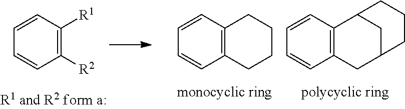

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

As used herein, the chemical group “D” refers to deuterium at an isotopic abundance greater than 25%, 35%, 50%, 60%, 70%, 80%, or 90%. In certain implementations, the chemical group “D” refers to deuterium at an isotopic abundance greater than 50%.

### Compositions

Most G protein-coupled receptors (GPCRs) are now recognized as multi-signal transducers. As such, agonists can act at a given receptor to preferentially activate one signal with minimal engagement of others, a property termed signal biasing. Biased agonists, then, could represent important advantages over non-biased agonists due to this signal selectivity, activating a specified therapeutic pathway while minimally evoking unnecessary or deleterious signaling. The pathway selectivity of biased agonists is thought to be established by the stabilization of specific conformation(s) of the agonist-receptor complex via a set of interactions that differ from those of unbiased (also called balanced) agonists. The signals/functions of a given GPCR that might be sought for selective activation are defined by the cell type, disease, and desired final physiological function.

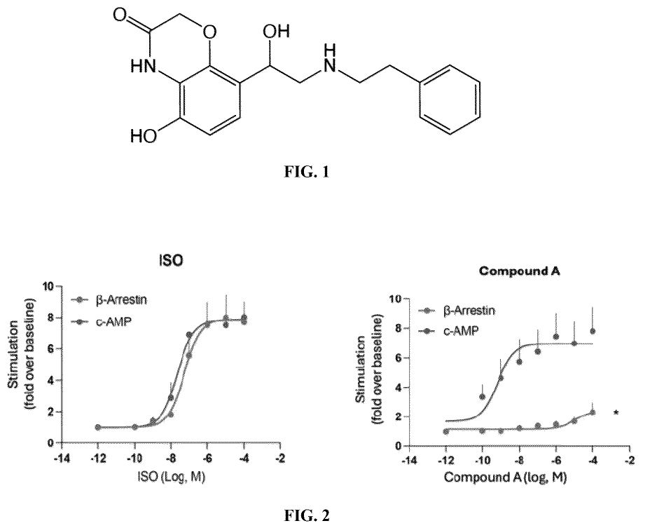

Agonists were sought that could stimulate the receptor to couple to Gs (thus stimulating cAMP) but fail to recruit the intracellular desensitizing protein beta-arrestin2. Desensitization is also termed tachyphylaxis and is a known property of current beta-agonists that are in use for treating asthma and chronic obstructive lung disease. Tachyphylaxis limits the therapeutic effectiveness of current beta-agonists. Utilizing in silico and cell-based approaches, a compound that acts as a biased beta-2 adrenergic receptor agonist has been synthesized. The compositions and methods disclosed herein comprise a beta-2 adrenergic agonist that is biased away from beta-arrestin and shows no tachyphylaxis, such as Compound A shown in FIG. 1. These Gαs-biased β₂AR agonists have decreased agonizing activity towards β-arrestin while still stimulating cAMP. The direction of the bias is away from beta-arrestin recruitment. In human airway smooth muscle cells, the balanced agonist isoproterenol (ISO) displays tachyphylaxis when measuring smooth muscle cell relaxation, while the beta-2 adrenergic agonists disclosed herein show no evidence of tachyphylaxis. The unique characteristics of Compound A pointed towards a beta-2 adrenergic receptor agonist that would not undergo desensitization, defined as a waning of the cellular response with continuous agonist exposure.

In some examples, a pharmaceutical composition can comprise a biased β₂-adrenergic receptor agonist with a structure according to Formula I:

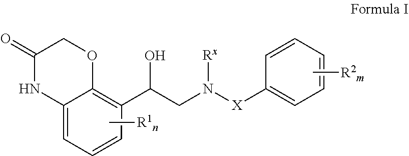

or a pharmaceutically acceptable salt thereof, wherein all variables are as defined further herein.

In some examples, n can be an integer from 0 to 3. In some examples, n can be 0. In some examples, n can be 1. In some examples, n can be 2. In some examples, n can be 3.

In some examples,

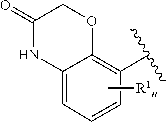

can be selected from:

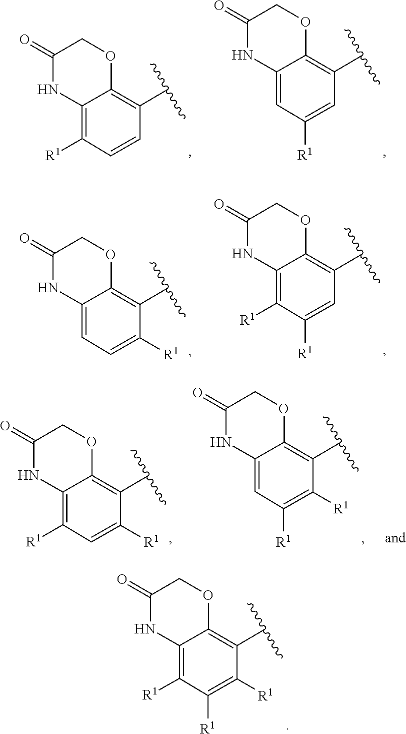

In some examples, m can be an integer from 0 to 5. In some examples, m can be 0. In some examples, m can be 1. In some examples, m can be 2. In some examples, m can be 3. In some examples, m can be 4. In some examples, m can be 5.

In some examples,

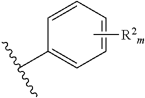

can be selected from:

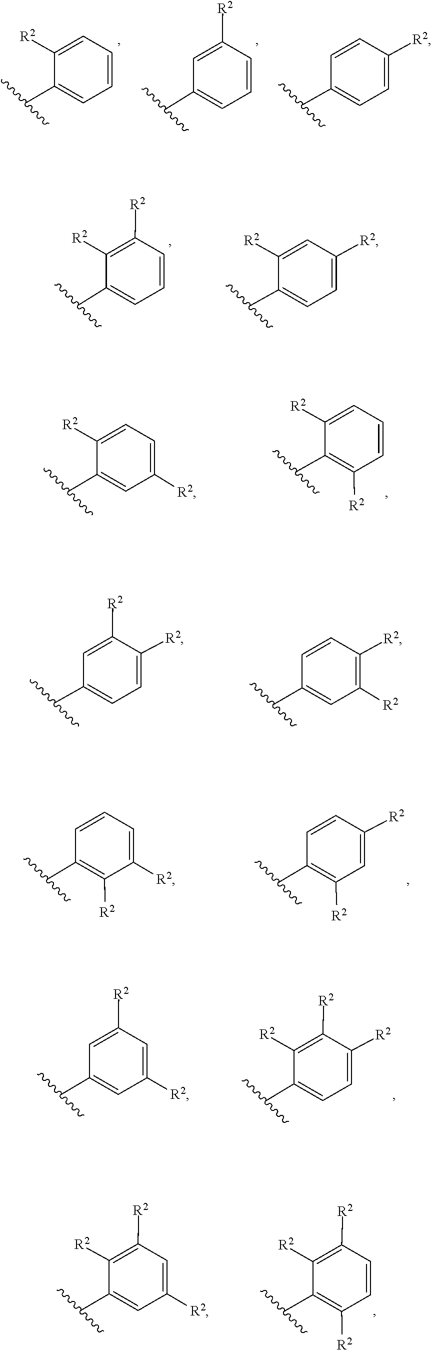

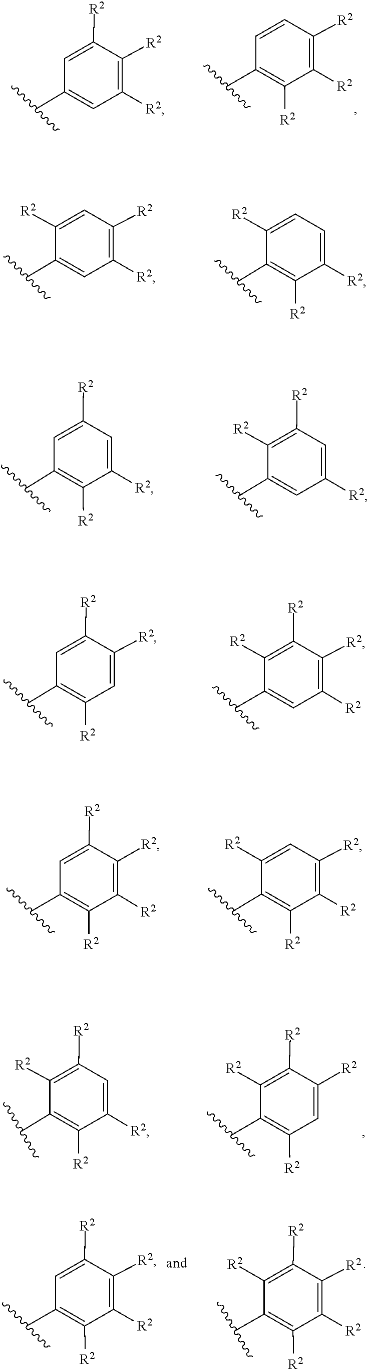

In some examples, R¹ can be each independently selected from the group consisting of -hydrogen, -hydroxyl, and OC₁₋₄ alkyl. In some examples, R¹ can be -hydrogen. In some examples, R¹ can be -hydroxyl. In some examples, R¹ can be OC₁₋₄ alkyl. In some examples, R² can be each independently selected from -hydrogen, -hydroxyl, and OC₁₋₄ alkyl. In some examples, R² can be -hydrogen. In some examples, R² can be -hydroxyl. In some examples, R² can be OC₁₋₄ alkyl.

In some examples, R¹ can be selected from methoxy, ethoxy, butoxy, isopropoxy, and tert-butoxy. In some examples, R² can be selected from methoxy, ethoxy, butoxy, isopropoxy, and tert-butoxy.

In some examples,

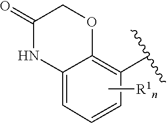

can be

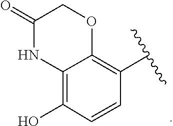 [SMILES: *c1ccc(O)c2c1OCC(=O)N2]

In some examples,

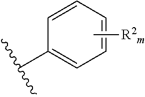

can be

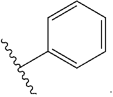 [SMILES: *c1ccccc1]

In some examples, X can be selected from —(CH₂)_{X1}— and —(CH₂)_{X2}O(CH₂)_{X2}—. In some examples, X can be —(CH₂)_{X1}—. In some examples, X can be —(CH₂)_{X2}O(CH₂)_{X2}—. In some examples, X1 can in each case be independently selected from 1-6. In some examples, X1 can be 1. In some examples, X1 can be 2. In some examples, X1 can be 3. In some examples, X1 can be 4. In some examples, X1 can be 5. In some examples, X1 can be 6.

In some examples, X2 can in each case be independently selected from 1-4. In some examples, X2 can be 1. In some examples, X2 can be 2. In some examples, X2 can be 3. In some examples, X2 can be 4.

In some examples, R^{X} can be H or C₁₋₄ alkyl. In some examples, R^{X} can be H. In some examples, R^{X} can be C₁₋₄ alkyl. In some examples, R^{X} can be selected from methyl, ethyl, n-propyl, isopropyl, n-butyl, isobutyl, sec-butyl, and t-butyl.

In some examples, the biased β₂-adrenergic receptor agonist of the pharmaceutical composition disclosed herein can be

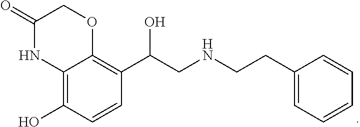 [SMILES: O=C1COc2c(C(O)CNCCc3ccccc3)ccc(O)c2N1]

### Methods of Administration

Disclosed are methods of treating an obstructive lung disease or condition in a subject without eliciting tachyphylaxis, comprising administering a therapeutically effective amount of a pharmaceutical composition comprising one or more biased β₂-adrenergic receptor agonists disclosed herein to a subject in need of treatment. In some examples, the obstructive lung disease or condition can be, e.g., asthma, chronic obstructive pulmonary disease (COPD), emphysema or bronchitis. The obstructive lung diseases and conditions can include any respiratory condition or disease, whether acute or chronic, characterized by impairment of airflow into and/or out of the lungs of a subject. Obstructive lung diseases and conditions include, e.g., asthma, chronic obstructive pulmonary disease (COPD), emphysema and bronchitis, as well as cystic fibrosis, bronchiectasis, bronchiolitis, and allergic bronchopulmonary aspergillosis.

In some examples, provided herein are methods of inducing bronchodilation in a subject, comprising administering a therapeutically effective amount of a pharmaceutical composition comprising one or more biased β₂-adrenergic receptor agonists disclosed herein to a subject in need of bronchodilation.

In some examples, provided herein are methods of relaxing airway smooth muscle (ASM) in a subject, comprising administering a therapeutically effective amount of a pharmaceutical composition comprising one or more biased β₂-adrenergic receptor agonists disclosed herein to a subject in need of ASM relaxation.

In some examples, provided herein are methods of treating or preventing bronchoconstriction or bronchospasm in a subject, comprising administering a therapeutically effective amount of a pharmaceutical composition comprising one or more biased β₂-adrenergic receptor agonists disclosed herein to a subject in need of treatment or prevention of bronchoconstriction or bronchospasm. Such bronchoconstriction or bronchospasm can be caused, e.g., by inhalation of a noxious compound such as smoke or a corrosive chemical; by a respiratory infection; or by anaphylaxis such as that caused by sepsis or an allergic reaction to a food (e.g., peanuts), a drug (e.g., penicillin), an insect sting or bite, pollen, mold, dust mites, latex, or other substances; or by other triggers of bronchoconstriction or bronchospasm. For example, the biased β₂-adrenergic receptor agonists can be administered to prevent (or treat) bronchospasm induced by exercise or air pollution. In another example, the biased β₂-adrenergic receptor agonists can be administered before or during placement of a breathing tube to prevent (or treat) bronchospasm induced by placement of the tube. The biased β₂-adrenergic receptor agonists disclosed herein can be administered to healthy individuals in situations in which it might be desirable to increase bronchodilation to improve oxygen uptake, e.g., in lower oxygen environments (such as several thousand feet above sea level) or to improve athletic performance.

In some examples, the biased β₂-adrenergic receptor agonists as disclosed herein can stimulate cAMP production through β₂-andrenergic receptor signaling with sub-nanomolar potency. In some examples, the therapeutically effective amount of a biased β₂-adrenergic receptor agonist as disclosed herein can be less than about 1 nM. In some examples, the therapeutically effective amount can be from about 0.1 nM-1 nM (e.g., from about 0.1 nM-0.5 nM, 0.5 nM-1 nM, 0.1 nM-0.25 nM, 0.25-0.5 nM, 0.5 nM-0.75 nM, 0.75 nM-1 nM, 0.1 nM-0.2 nM, 0.2 nM-0.3 nM, 0.3 nM-0.4 nM, 0.4 nM-0.5 nM, 0.5 nM-0.6 nM, 0.6 nM-0.7 nM, 0.7 nM-0.8 nM, 0.8 nM-0.9 nM, or 0.9 nM-1 nM). In some examples, the therapeutically effective amount can be about 0.25 nM.

### Pharmaceutical Formulations

The pharmaceutical compositions comprising one or more biased β₂-adrenergic receptor agonists disclosed herein can also comprise one or more of a carrier, diluent and excipient (e.g., a pharmaceutically acceptable carrier, diluent, or excipient), depending on the identity of the compound. By “pharmaceutically acceptable” is meant a material that is not biologically or otherwise undesirable, e.g., the material may be administered to an individual along with the compound without causing any undesirable biological effects or interacting in a deleterious manner with any of the components of the pharmaceutical composition in which it is contained. Conventional pharmaceutical practice may be employed to provide suitable formulations or compositions to administer such compositions to subjects. The terms specifically exclude cell culture medium. Suitable diluents (for both dry and liquid pharmaceutical formulations) are well known to those skilled in the art and include saline, buffered saline, dextrose (e.g., 5% dextrose in water), water, glycerol, ethanol, propylene glycol, polysorbate 80 (Tween-80™), poly(ethylene) glycol 300 and 400 (PEG 300 and 400), PEGylated castor oil (e.g. Cremophor EL), poloxamer 407 and 188, a cyclodextrin or a cyclodextrin derivative (including HPCD ((2-hydroxypropyl)-cyclodextrin) and (2-hydroxyethyl)-cyclodextrin; see, e.g., U.S. patent application publication 20060194717).

Carriers are compounds and substances that improve and/or prolong the delivery of an active ingredient to a subject in the context of a pharmaceutical formulation. Carriers can serve to prolong the in vivo activity of a drug or slow the release of the drug in a subject, using controlled-release technologies. Carriers can also decrease drug metabolism in a subject and/or reduce the toxicity of the drug. Carriers can also be used to target the delivery of the drug to particular cells or tissues in a subject. Common carriers (both hydrophilic and hydrophobic carriers) include fat emulsions, lipids, PEGylated phospholids, liposomes and lipospheres, microspheres (including those made of biodegradable polymers or albumin), polymer matrices, biocompatible polymers, protein-DNA complexes, protein conjugates, erythrocytes, vesicles and particles.

Excipients included in a pharmaceutical composition have different purposes depending, for example on the nature of the drug, and the mode of administration. Examples of generally used excipients include, without limitation: stabilizing agents, solubilizing agents and surfactants, buffers and preservatives, tonicity agents, bulking agents, lubricating agents (such as talc or silica, and fats, such as vegetable stearin, magnesium stearate or stearic acid), emulsifiers, suspending or viscosity agents, inert diluents, fillers (such as cellulose, dibasic calcium phosphate, vegetable fats and oils, lactose, sucrose, glucose, mannitol, sorbitol, calcium carbonate, and magnesium stearate), disintegrating agents (such as crosslinked polyvinyl pyrrolidone, sodium starch glycolate, cross-linked sodium carboxymethyl cellulose), binding agents (such as starches, gelatin, cellulose, methyl cellulose or modified cellulose such as microcrystalline cellulose, hydroxypropyl cellulose, sugars such as sucrose and lactose, or sugar alcohols such as xylitol, sorbitol or maltitol, polyvinylpyrrolidone and polyethylene glycol), wetting agents, antibacterials, chelating agents, coatings (such as a cellulose film coating, synthetic polymers, shellac, corn protein zein or other polysaccharides, and gelatin), preservatives (including vitamin A, vitamin E, vitamin C, retinyl palmitate, and selenium, cysteine, methionine, citric acid and sodium citrate, and synthetic preservatives, including methyl paraben and propyl paraben), sweeteners, perfuming agents, flavoring agents, coloring agents, administration aids, and combinations thereof.

The pharmaceutical compositions disclosed herein can be formulated for pulmonary administration, whether for nasal or buccal inhalation. The unit dosage of the pharmaceutical composition can be conveniently delivered in the form of an aerosol spray from a pressurized pack or a nebulizer, or via a vaporizer. The pharmaceutical compositions can also be delivered as a formulated powder and the powder composition can be inhaled with the aid of an insufflation powder inhaler device. One example of a delivery system for inhalation is a metered dose inhalation (MDI) aerosol, which can be formulated as a dry suspension or wet solution of a pharmaceutical composition disclosed herein in a suitable propellant, such as a fluorocarbon solvent or a hydrocarbon solvent. Suitable fluorocarbon solvents include HFA-134a (1,1,1,2-tetrafluoroethane), HFA-227ea (1,1,1,2,3,3,3-heptafluoropropane), HFA-152a (1,1-difluoroethane) and combinations thereof. For propellant formulations, the propellant can be present in an amount that is at least 5% by weight, at least 10% by weight, at least 25% by weight, at least 50% by weight at least 75% by weight, or at least 90% by weight, relative to the entire formulation. In some examples, the propellant is present in an amount from 80-99.9% by weight, or from 90-99.9% by weight, relative to the entire formulation. The propellant formulation can also include one or more stabilizing excipients, such as ethanol and oleic acid. When ethanol is used, it may be present in an amount from 0.5-10% by weight, from 1-5% by weight, or from 5-10% by weight, relative to the entire formulation. The propellant formulations can further include one or more surfactants, for instance in an amount from 0.1-2.5% by weight, or from 0.2-1.5% by weight, relative to the entire formulation.

The biased β₂-adrenergic receptor agonists can be in particulate form to enhance delivery to the lung. For example, the biased β₂-adrenergic receptor agonists can be provided with a particle from 0.1-10 μm, from 0.1-5 μm, from 0.5-2.5 μm, from 1-5 μm, from 2.5-5 μm, or from 1-2.5 μm. In the case of dry powder formulations, the disclosed compounds can be provided in an amount from 1-50% by weight, from 1-50% by weight, from 5-50% by weight, from 1-25% by weight, from 10-25% by weight, from 15-50% by weight, or from 25-50% by weight relative to the total weight of the formulation. The dry powder formulations can further include a powdered matrix material, for example a polyol or carbohydrate, e.g., sorbitol, mannitol, xylitol, glucose, arabinose, lactose, maltose, saccharose, dextrose and mixtures thereof. The compositions can further include a surfactant, for example in an amount from 0.1-10% by weight, from 0.1-5% by weight, 1-10% by weight, from 1-5% by weight, 2.5-10% by weight, or from 2.5-5% by weight. Suitable surfactants include lecithin, phospholipids derivatives such as phosphatic acids, phosphatidyl choline (saturated and unsaturated), phosphatidyl ethanol amine, phosphatidyl glycerol, phosphatidyl serine, phosphatidyl inositol, dioleoylphosphatidylcholine, dimyristoyl phosphatidylcholine, dipalmitoylphosphatidylcholine, distearoyl phosphatidylcholine, diarachidoyl phosphatidylcholine, dibenoyl phosphatidylcholine, ditricosanoyl phosphatidylcholine, dimiristoylphosphatidylethanol-amine, dipalmitoyl-dilignoceroylphatidylcholine, phosphatidylethanoalamine, pipalmitoleoylphasphatidylethanol-amine, distearoyl-phosphatidylethanolamine, dimyristoylphosphatidylglycerol, dipalmitoylphosphatidyl glycerol, dipalmitolcoylphosphatidylglycerol, and mixtures thereof.

In addition, any other appropriate route for administration can be employed, for example, but not limited to, intravenous, parenteral, transbuccal, transdermal, transcutaneous, subcutaneous, intranasal, aerosol, or oral administration. Therapeutic formulations can be in the form of liquid solutions or suspensions; for example, for oral administration, formulations can be in the form of tablets or capsules; for intranasal formulations, in the form of powders, nasal drops, or aerosols; for transdermal formulations, in the form of creams or distributed onto patches to be applied to the skin.

Effective amounts of the biased β₂-adrenergic receptor agonists disclosed herein in a pharmaceutical formulation will vary depending on the compound being used and the condition or disease being treated, as well as factors such as age of the subject and other medications being taken. Effective dosages will typically be set by an attending physician as is well known in the art. However, the concentration of the biased β₂-adrenergic receptor agonists delivered to a subject in a unit dose will generally range from about 0.05 mg to about 100 mg, or a value within this range. The combinations can be administered in combinations and/or in combination with one or more other agent (e.g. but not limited to a beta-agonist such as albuterol, levalbuterol, metaproterenol, pirbuterol, salmeterol, formoterol, arformoterol, indacaterol, terbutaline, olodaterol, abediterol, or vilanterol). In one non-limiting example provided herein, the biased β₂-adrenergic receptor agonists can be administered with either isoproterenol or chloroquine; thus, under some circumstances, it can be appropriate to administer either (or both) of these agents combination with the biased β₂-adrenergic receptor agonists disclosed herein to a subject in need thereof.

### Examples

The following examples are put forth so as to provide those of ordinary skill in the art with a complete disclosure and description of how the compounds and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Using in silico docking and synthesis, it was discovered that 5-hydroxy-8-(1-hydroxy-2-(phenethylamino)ethyl)-2H-benzo[b][1,4]oxazin-3 (4H)-one (called Compound A, FIG. 1) is a potent stimulator of CAMP in cells transfected to express the human beta-2 adrenergic receptor. No stimulation was observed in non-transfected cells. The known agonist isoproterenol (ISO) was used as a positive control. The potency of Compound A for stimulating cAMP was 0.25 nanomolar.

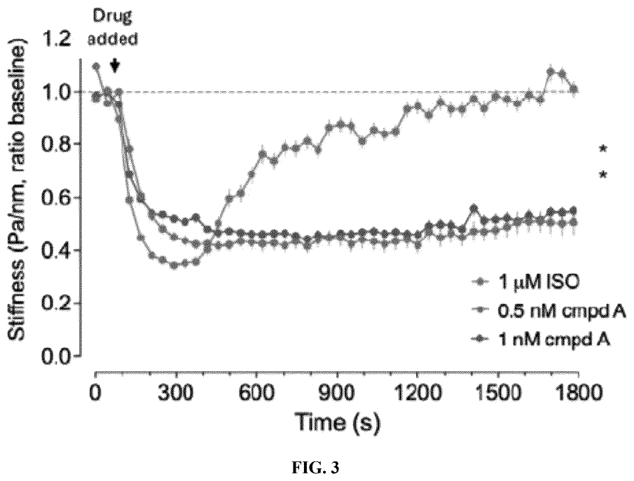

To directly test whether Compound A displayed desensitization, human airway smooth muscle cells (HASM) were used, and stiffness was measured using magnetic twisting cytometry. The beta-2 adrenergic receptors on HASM are the targets of beta-agonist bronchodilators used for treating obstructive airway diseases, by relaxing HASM and opening airways. So, the cell type and the physiological response measured in these experiments are relevant. The results are shown in FIG. 3. The ISO response wanes over time, consistent with the known desensitization function of beta-arrestin, which is stimulated by ISO in a balanced fashion (see FIG. 2). In marked contrast, the relaxation response to Compound A, which is biased away from beta-arrestin, remained stable throughout the study period.

The plots in FIG. 2 show that Compound A stimulates cAMPto the same extent as the control agonist ISO. However, Compound A minimally activates recruitment of beta-arrestin2. Thus, while ISO is a “balanced agonist” (the CAMP and beta-arrestin curves are superimposed), Compound A is a “biased agonist”, being biased towards the CAMP pathway and away from the beta-arrestin pathway.

Taken together, a unique compound has been synthesized and has been shown that it acts as a beta-2 adrenergic receptors as a biased agonist, with the bias being away from beta-arrestin activation. Thus, Compound A shows no desensitization/tachyphylaxis, which would be a therapeutic advantage over other beta-agonists.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A pharmaceutical composition, comprising a biased β₂-adrenergic receptor agonist according to Formula I: 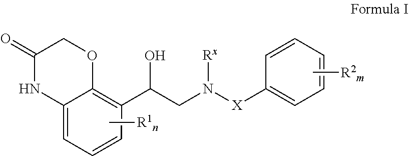
or a pharmaceutically acceptable salt thereof, wherein:
n is an integer from 0 to 3;
m is an integer from 0 to 5;
R¹ is each independently selected from -hydrogen, -hydroxyl, and OC₁₋₄ alkyl;
R² is each independently selected from -hydrogen, -hydroxyl and OC₁₋₄ alkyl;
X is selected from —(CH₂)_{X1}— and —(CH₂)_{X2}O(CH₂)_{X2}—, wherein X1 is 1-6, X2 is in each case independently selected from 1-4; and
R^{X} is H or C₁-4 alkyl.

2. The pharmaceutical composition of claim 1, wherein n is 1.

3. The pharmaceutical composition of claim 1, wherein m is 0.

4. The pharmaceutical composition of claim 1, wherein 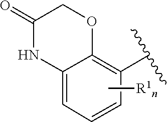
is

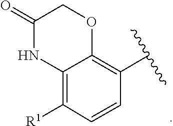

5. The pharmaceutical composition of claim 1, wherein R¹ is hydroxyl.

6. The pharmaceutical composition of claim 1, wherein X is —(CH₂)_{X1}—.

7. The pharmaceutical composition of claim 6, wherein X1 is 2.

8. The pharmaceutical composition of claim 1, wherein the biased β₂-adrenergic receptor agonist is

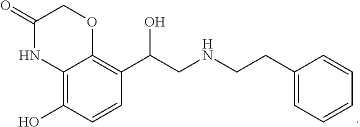 [SMILES: O=C1COc2c(C(O)CNCCc3ccccc3)ccc(O)c2N1]

9. A method of treating obstructive lung disease without eliciting tachyphylaxis in a subject in need thereof, comprising administering a therapeutically effective amount of the pharmaceutical composition according to claim 1.

10. The method of claim 9, wherein the obstructive lung disease comprises asthma, chronic obstructive pulmonary disease (COPD), emphysema, or bronchitis.

11. The method of claim 9, further comprising administering at least one additional therapeutic agent.

12. The method of claim 9, wherein the therapeutically effective amount comprises from about 0.1-1 nM.

13. A method of relaxing airway smooth muscle (ASM) without eliciting tachyphylaxis in a subject in need thereof, comprising administering a therapeutically effective amount of the pharmaceutical composition according to claim 1.

14. A method of inducing bronchodilation without eliciting tachyphylaxis in a subject in need thereof, comprising administering a therapeutically effective amount of pharmaceutical composition according to claim 1.

15. A method of treating bronchoconstriction or bronchospasm without eliciting tachyphylaxis in a subject in need thereof, comprising administering a therapeutically effective amount of the pharmaceutical composition according to claim 1.

16. A method of treating obstructive lung disease without eliciting tachyphylaxis in a subject in need thereof, comprising administering a therapeutically effective amount of 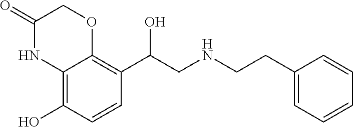 [SMILES: O=C1COc2c(C(O)CNCCc3ccccc3)ccc(O)c2N1]
or a pharmaceutically acceptable salt thereof.

17. The method of claim 16, wherein the obstructive lung disease comprises asthma, chronic obstructive pulmonary disease (COPD), emphysema, or bronchitis.

18. The method of claim 16, further comprising administering at least one additional therapeutic agent.

19. The method of claim 16, wherein the therapeutically effective amount comprises from about 0.1-1 nM.

20. The method of claim 16, wherein the therapeutically effective amount is about 0.25 nM.
